(19) United States
(12) Patent Application Publication
(10) Pub. No.: US 2020/0108017 A1
(43) Pub. Date: Apr. 9, 2020

(54) TWO SPEED MONOLITHIC SYSTEM FOR CONTROLLED RELEASE OF DRUGS

(71) Applicant: MATRIPHARM INTERNATIONAL INC., Laval (CA)

(72) Inventors: Tien Canh LE, Montreal (CA); Mircea-Alexandru MATEESCU, Montreal (CA)

(21) Appl. No.: 16/704,829

(22) Filed: Dec. 5, 2019

Related U.S. Application Data
(63) Continuation of application No. 15/637,592, filed on Jun. 29, 2017, now abandoned.
Application No. 15/637,592 is a continuation of application No. 14/002,499, filed on Aug. 30, 2013, now abandoned.
(60) Provisional application No. 61/447,765, filed on Mar. 1, 2011.

(51) Int. Cl. A61K 9/20; A61K 31/192; A61K 31/405; A61K 31/616; A61K 45/06

(52) U.S. Cl. CPC A61K 9/2059; A61K 9/2027; A61K 9/205; A61K 9/2054; A61K 31/192; A61K 9/2013; A61K 31/616; A61K 9/2009; A61K 9/2018; A61K 45/06; A61K 31/405

(57) ABSTRACT

The present document describes a monolithic tablet dosage form for delivery of an active ingredient at two different release rates comprising a carboxyl polymer complexed with a multivalent cation and a disintegrating agent for a first initial fast release of the active ingredient, and a modulating agent for a second sustained release of the active ingredient. Also described are processes for preparing the carboxyl polymer complexed with a multivalent cation, and carboxyl polymer made from the process.

### RELATED APPLICATIONS

This application is filed under 37 CFR 1.53(b) as a continuation application. This application claims priority of U.S. patent application Ser. No. 15/637,592 filed on Jun. 29, 2017, which claims priority of U.S. patent application Ser. No. 14/002,499 filed on Aug. 30, 2013, which is a US National Phase application under 35 USC § 371 of PCT/CA2012/000180, filed Feb. 28, 2012, which claims priority to U.S. Provisional Application No. 61/447,765, filed Mar. 1, 2011, the specifications of which are hereby incorporated by reference in their entireties.

### BACKGROUND

### (a) Field

The subject matter disclosed generally relates to dosage forms for delivery of active ingredients at two different release rates. More specifically, the dosage form comprises a carboxyl polymer complexed with a multivalent cation and a disintegrating agent. Also disclosed are processes for preparing the carboxyl polymer complexed with a multivalent cation, and a carboxyl polymer made from the process.

### (b) Related Prior Art

Non-steroidal anti-inflammatory drugs (NSAIDs) are the most widely prescribed for inflammatory symptoms. NSAIDs such as ibuprofen, naproxen, aspirin, etc. are drugs with analgesic, antipyretic and anti-inflammatory effects. The main advantage of NSAIDs is that (unlike opioids) they do not cause sedation, respiratory depression or addiction. Certain NSAIDs have become accepted as relatively safe and rescheduled (e.g. ibuprofen) to allow availability over-the-counter.

Chronic inflammation is closely related to many diseases and conditions, often associated with aging. It is also associated with many pathologies such as arthritis, gastric reflux disease, colitis, some forms of cancer, Alzheimer's disease, immune dysfunctions and/or cardiovascular diseases. There is a great need for effective therapy to prevent or reduce inflammatory conditions, especially treatments that will be simple, effective and will have little or no adverse side effects. Frequently, patients requiring long term NSAID therapy are at risk of developing peptic ulcers and ulcer-related upper gastro-intestinal complications, NSAIDs are a well-defined cause of these complications. In addition, recent evidence suggests that NSAIDs may increase cardiovascular risks, particularly in patients with a history of hypertension, diabetes or renal failure.

In the largest study in the field to date, Julia Hippisley-Cox and Carol Coupland, (2005. Br. Med. J., 330, 1366-1369) found that certain patients who had NSAIDs prescribed have a higher risk of heart attack, compared with those who had not taken these drugs in the previous three years. For ibuprofen, the risk of heart attack increased by 24%, and for diclofenac it rose to 55%.

The most significant findings concerned the drugs ibuprofen, diclofenac and rofecoxib. In terms of «numbers needed to harm», for the patients in the age group of 65 and over taking diclofenac, one out of 521 patients was likely to suffer a first-time heart attack. For rofecoxib, it was one out of 695 patients, and for ibuprofen, one out of 1005 patients.

It is difficult for health care practitioners to suggest safe NSAIDs for patients requiring long-term NSAID therapies. Various strategies have been used to reduce the risk of NSAID-related gastro-intestinal or cardiovascular complications. The use of slow-release formulations seems appropriate to reduce side effects caused by the NSAIDs. Furthermore, the combination of anti-acids (e.g. proton pump inhibitor such as Omeprazole) with NSAIDs to reduce the risk of NSAID-related ulcers, gastrointestinal or cardiovascular complications, is of interest.

As described above, it is of interest to reduce risks of peptic ulcers and ulcer-related gastro-intestinal complications, and in certain cases, the risk of developing cardiovascular disease. The use of slow-release formulations seems appropriate to reduce side effects related to the NSAIDs.

One aim of the present invention is to provide a process to produce a powder complex obtained by ionic complexation of linear polyanions via multivalent cations. This complex is used as hydrophilic stabilizer that is mechanically resistant and able to absorb gastric or intestinal fluids. When this stabilizer is associated with an insoluble disintegrating agent, both form a matrix which, under monolithic tablet form, is able to deliver the active principle in a controlled manner at different speeds.

Another aim of the present invention is to provide a pharmaceutical composition to control release of NSAIDs in two speeds: a first fast release and, then, a second slow release. The fast release provides initially an effective dose of active principle, whereas the subsequent slow release of active principle lasts over several hours.

### SUMMARY

A method to prepare a cationic-carboxyl polymer complex that is stable in any pH and able to hydrate and absorb the biological fluids. When associated with a suitable disintegrating agent, the both form the matrix. Used under monolithic tablet dosage form, this matrix can deliver drugs, particularly non-steroidal anti-inflammatory (NSAIDs) at two different speeds: fast and slow release. The first consists in delivering initially a burst dose over a period of time of 1-2 h, followed by the second sustained dose lasting over at least 6 h after the first effective dose.

According to an embodiment, there is provided a dosage form for delivery of an active ingredient at two different release rates comprising:
a carboxyl polymer complexed with a multivalent cation; and
a disintegrating agent, for a first initial fast release of the active ingredient; and
a modulating agent, for a second sustained release of the active ingredient.

According to an embodiment, there is provided a dosage form for delivery of an active ingredient where the matrix is monolithic and comprises:
a carboxyl polymer complexed with a multivalent cation; and
a disintegrating agent, for a first initial release of the active ingredient;
a modulating agent, for a second sustained sustained release of the active ingredient.

The carboxyl polymer complexed with multivalent cations may be chosen from an anionic polysaccharide, natural polysaccharide, and a synthetic carboxyl polymer or combination thereof.

The anionic polysaccharide may be chosen from carboxymethylcellulose, carboxymethylstarch, carboxyl high amylose-starch, carboxymethyl-chitosan, and combinations thereof.

The natural polysaccharide may be chosen from a pectin, hyaluonan (hyaluronic acid), xanthane, gellan, alginate, and combinations thereof.

The synthetic carboxyl polymer may be chosen from a carbomer (polyacrylic acid) or a cross-linked carbomer.

The multivalent cation may be a divalent cation.

The divalent cation may be chosen from calcium (Ca²⁺), magnesium (Mg²⁺), copper (Cu²⁺), zinc (Zn²⁺), iron (Fe²⁺), and combinations thereof.

The divalent cation is preferably calcium (Ca²⁺).

The carboxyl polymer complexed with a multivalent cation may be present in a concentration of about 1% to about 75%.

The carboxyl polymer complexed with a multivalent cation may be present in a concentration of about 2% to about 50%.

The carboxyl polymer complexed with a multivalent cation may be present in a concentration of about 5% to about 40/®.

The disintegrating agent may be chosen from a povidone, a povidone derivative, a crospovidone, a crosslinked sodium carboxymethyl cellulose, cross-link starch, a cross-linked starch glycolate, a cellulose, a cellulose derivative, an alginate, a soy polysaccharide, or combinations thereof.

The crospovidone is preferably a crosslinked povidone.

The cross-linked starch is preferably sodium starch glycolate.

The disintegrating agent may be present in a concentration of about 1% to about 75%.

The disintegrating agent may be present n a concentration of about 5% to about 50%.

The disintegrating agent may be present in a concentration of about 10% to about 40%.

The modulating agent may be chosen from polyvinylpyrollidone, a chondroitin, a hyaluronate, or combinations thereof.

The modulating agent may comprise a molecule containing an amino group.

The modulating agent may be chosen from molecules possessing a positive charge such as glucosamine and its salts, choline, lecithin, phosphatidylcholine or amino acids. The amino acids may be lysine, tyrosine, glutamine, valine, phenylalanine, asparagine, arginine, leucine, isoleucine, tryptophan, histidine, methionine, threonine, serine, glycine, proline, glutamic acid, aspartic acid, cysteine, selenocysteine, and alanine, etc, or combination thereof.

The modulating agent is preferably glucosamine or its salts.

The modulating agent may be present in a concentration of about 1% to about 75%.

The modulating agent may be present in a concentration of about 5% to about 50%.

The modulating agent may be present in a concentration of about 10% to about 40%.

The dosage form may further comprise a binder agent.

The binder agent may be chosen from a microcrystalline cellulose, hydroxypropyl methylcellulose, hydroxypropylcellulose, ethylcellulose, methyl cellulose, amylose, noncrosslinked polyvinylpyrrolidone or combinations thereof.

The povidone preferably has a K-value between 15 and 90.

The binder agent may be present in a concentration of about 0.1 to about 15%.

The binder agent may be present in a concentration of about 0.5% to about 15%.

The dosage form may further comprise a lubricating agent.

The lubricating agent may be chosen from talc, silica, a fat, sorbitol, a polyethylene glycol (PEG), or combinations thereof.

The fat may be chosen from a vegetable stearine, magnesium stearate, stearic acid of combinations thereof.

The lubricating agent may be present in a concentration of about 0.1% to about 3.5%.

The dosage form may further comprise an active principle.

The active principle may be chosen from a non-steroidal anti-inflammatory drug (NSAID) and an antihistaminic agent.

The non-steroidal anti-inflammatory drug (NSAID) may be chosen from ibuprofen, naproxen, benoxaprofen, flurbiprofen, fenoprofen, fenbuprofen, ketoprofen, ioxoprofen, pranoprofen, carprofen, oxoprofen, microprofen, tioxaprofen, suproprofen, alminoprofen, fluprofen, aspirin, diflunisal, salsalate, olsalazine, sulfasalazine, indomethacin, sulindac, etodolac, ketorolac, diclofenac, mefenamic, meclofenamic, flufenamic, tolfenamic, celecoxib, valdecoxib, rofecoxib, rterocoxib or the combination thereof.

According to another embodiment, there is provided a process for the preparation of a carboxyl polymer complexed with a multivalent cation comprising:
incubating a carboxyl polymer with an excess of an ionic compound comprising a multivalent cation, for a time sufficient for complexation; and
precipitating the carboxyl polymer complexed with a multivalent cation with organic solvents or spray-drying to obtain powders.

The process may further comprise sieving the dried carboxyl polymer complexed with a multivalent cation.

The ionic compound may be chosen from CaCl₂, MgCl₂, CuCl₂, ZnCl₂, FeCl₂, or combinations thereof.

The carboxyl polymer may be chosen from an anionic polysaccharide, natural polysaccharide, and a synthetic carboxyl polymer or combination thereof.

The anionic polysaccharide may be chosen from carboxymethylcellulose, carboxymethylstarch, carboxyl high amylose-starch, carboxymethyl-chitosan, and combinations thereof.

The natural polysaccharide may be chosen from a pectin, hyaluronan (hyaluronic acid), xanthane, gellan, alginate, and combinations thereof.

The synthetic carboxyl polymer may be chosen from a carbomer (polyacrylic acid) or a cross-linked carbomer.

According to another embodiment, there is provided a carboxyl polymer complexed with a multivalent cation prepared by the present process.

The following terms are defined below.

The term “monolithic” is intended to mean a system with an unchanging and homogeneous uniform structure with no individual local variation.

The term “two release rates” in intended to mean that the monolithic system of the present invention will initially release an active ingredient at a first initial rate, followed by a second release of the active ingredient with a different second rate.

The term “first effective dose” or “first initial release” is intended to mean the dose of the active ingredient that is released after initial administration of the dosage form. It may be a “fast” dose released rapidly after administration of the dosage form.

The term “second effective dose” is intended to mean the dose of the active ingredient that is released after first dose of the ingredient. It may be a “slower” dose released rapidly after administration of the dosage form, and it may span several hours as a sustained release.

The term “fast release” is intended to mean any period of time between 5 minutes to 2 hours, preferably any period of time between 15 minutes to 1 hour, more preferably about 30 minutes.

The term “sustained release” or “slow release” is intended to mean any period of time of at least 6 hours and more, it varies depending on the quantity of dosage released initially, the more the initial release is important (i.e. 50% of the dosage) the more the time of release of the second rate is short (i.e. 6 hours), the more the total amount of dosage to be released is important (i.e. 600 mg of the dosage) the more the time of release of the second rate is long (i.e. 14 hours).

The term “dosage form” is intended to mean pill, tablet, or capsule, or suppository (rectal, vaginal) device for the delivery of an active ingredient.

Features and advantages of the subject matter hereof will become more apparent in light of the following detailed description of selected embodiments, as illustrated in the accompanying figures. The subject matter disclosed and claimed is capable of modifications in various respects, all without departing from the scope of the claims. Accordingly, the drawings and the description are to be regarded as illustrative in nature, and not as restrictive. The full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIG. 1 illustrates FTIR spectra of calcium and sodium CarboxymethylStarch tablets before (A) and after incubation in simulated gastric fluid (B) for 2 h at 37° C.

FIG. 2 illustrates X-ray diffractograms of non-modified (native) starch and of sodium and calcium CarboxymethylStarch.

FIG. 3 illustrates a release pattern of ibuprofen (600 mg) from tablets based on calcium CarboxymethylStarch. The profile is characterized by two different speeds, first fast (releasing about of 30%) and then sustained release of remaining dose for a period over 6 h. The release kinetics were followed in 1 L of simulated gastric fluid (pH 1.5) for 2 h, and then in 1 L of simulated intestinal fluid (pH 6.8, at 37° C. and 100 rpm), with a dissolution device Distek (Apparatus 2).

FIG. 4 illustrates release profiles of ibuprofen (600 mg) from tablets based on calcium and sodium CarboxymethylStarch. The release kinetics were followed in 1 L of simulated gastric fluid (pH 1.5) for 2 h, and then in 1 L of simulated intestinal fluid (pH 6.8, at 37° C. and 100 rpm), with a dissolution device Distek (Apparatus 2).

FIG. 5 illustrates release profiles of ibuprofen (600 mg) dissolution from tablets based-on calcium CarboxymethylStarch at various degrees of substitution. The release kinetics were followed in 1 L of simulated gastric fluid (pH 1.5) during 2 h and then in 1 L of simulated intestinal fluid (pH 6.8), at 37° C. and 100 rpm, with a dissolution device Distek (Apparatus 2).

FIG. 6 illustrates pharmacokinetic profiles of Ibuprofen (400 mg×1) formulated with new controlled release monolithic tablets based on Calcium CarboxymethylStarch excipients compared with commercial Motrin® (Ibuprofen 200 mg×3) immediate release tablets, in a study on Beagle dogs.

FIG. 7 illustrates cumulative area under the curve (AUC₀₋₂₄) of Motrin® and Calcium CarboxymethylStarch monolithic tablet.

FIG. 8 illustrates FTIR spectra of calcium and sodium Carboxymethylcellulose tablets before (A) and after (B) incubation in simulated gastric fluid during 2 h at 37° C.

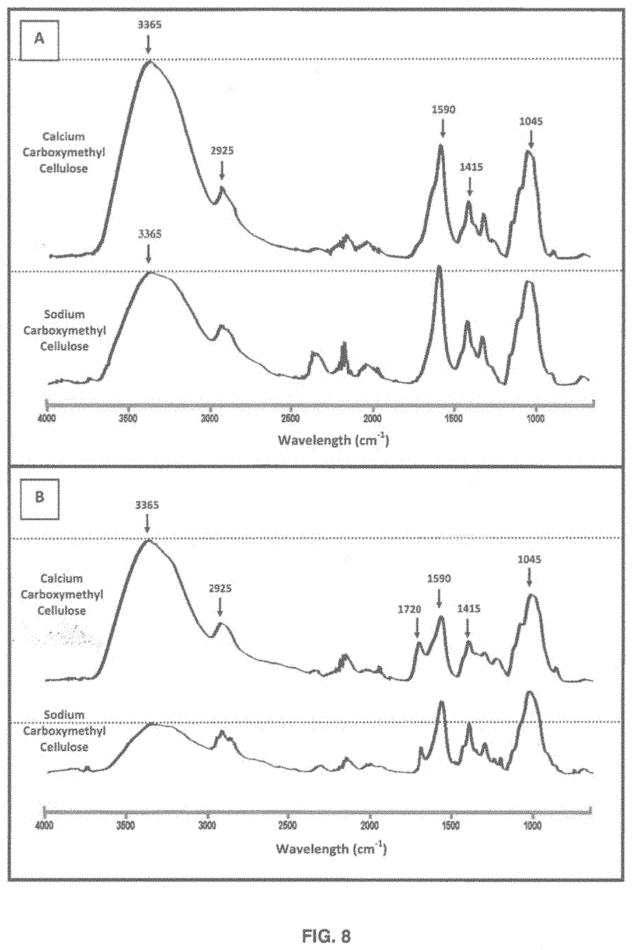

FIG. 9 illustrates X-ray diffractograms of non-modified (native) cellulose and of sodium and calcium carboxymethylcellulose.

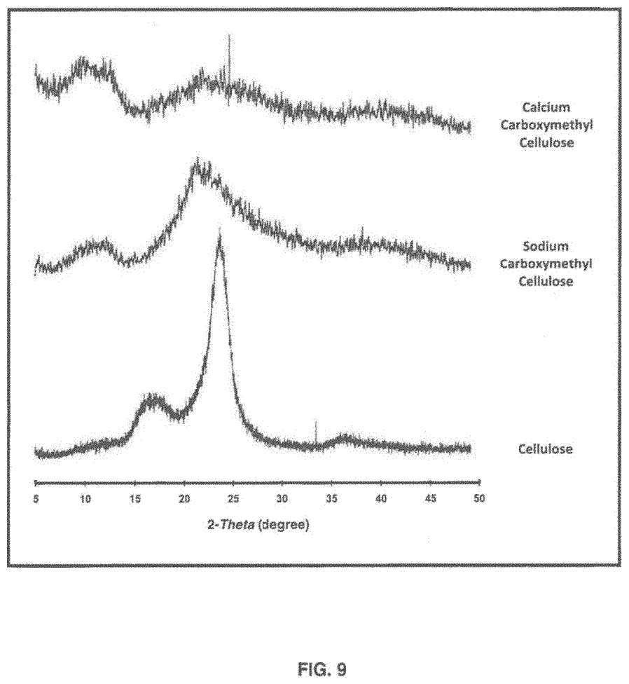

FIG. 10 Ilustrates kinetic profiles of ibuprofen (600 mg) dissolution from tablets based on calcium and sodium Carboxymethylcellulose. The release kinetics were followed in 1 L of simulated gastric fluid (pH 1.5) for 2 h, and then in 1 L of simulated intestinal fluid (pH 6.8), at 37° C. and 100 rpm, with a dissolution device Distek (Apparatus 2).

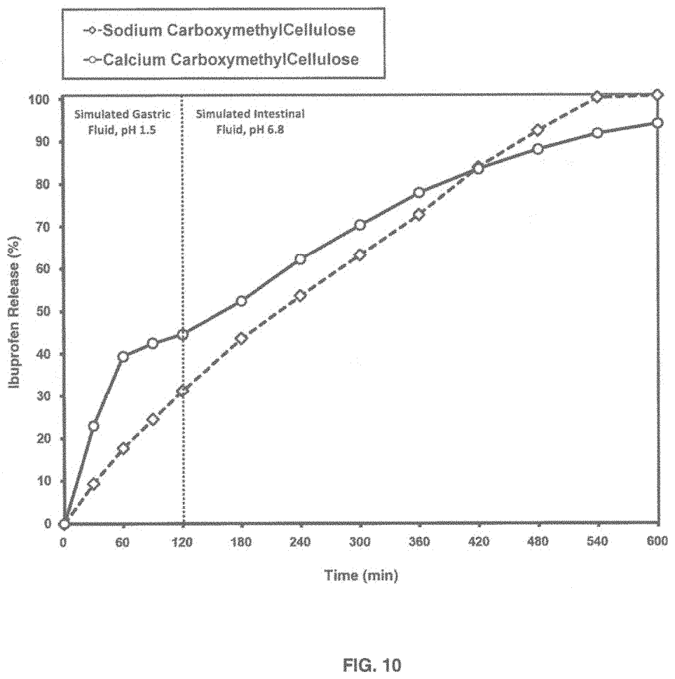

### DETAILED DESCRIPTION OF THE PREFERRED EMBODIMENTS

In embodiments there is disclosed a process to produce an ionic complexation of a hydrophilic stabilizer which is associated with an insoluble disintegrating agent to form a matrix. According to one embodiment of the present invention, the polymers used possess carboxyl groups that can interact with multivalent cations by complexation or sequestration. This phenomenon is also known in some cases as ionotropic gelation or ionic stabilization.

As used herein, the terms «carboxyl polymer complexed with multivalent cations» or «ionotropic gelation of carboxyl polymer» or «ionic stabilized carboxyl polymer» or represent polymers having carboxylate groups which are mainly under sequestration or complexation form with multivalent cations.

Used under monolithic tablet dosage form, the carboxyl polymer complexed with multivalent cations of the present invention are found to possess a greater hydration or water absorption capacity than other polymeric forms, such as sodium or potassium salt or acid forms, not complexed with multivalent cations. Furthermore, the ionic-polymer complex of the present invention is mechanically stable at any pH value (gastric acid or intestinal media). When associated with a disintegrating agents (disintegrants), the carboxyl polymer complexed with multivalent cations of the present invention and the desintegrating agents formed a matrix able to deliver an active principle at different speeds: a first fast release followed by a sustained release for at least 6 h or more.

Without being bound to theory, it is believed that polymers according to the present invention possessing carboxyl groups generate a stable matrix in gastric acid (pH<3). Initially, under sodium or potassium salt forms, there a protonation of carboxylate (—COO⁻Na⁺) into carboxylic acid (—COOH) groups which are stabilized via several polar interactions limiting or decreasing thus the hydration (Assaad and Mateescu, 2010, Int. J. Pharma, 394, 75-84). The release profile observed with those matrices is single speed (one speed).

In contrast, when the carboxyl polymer is complexed with multivalent cations as calcium (Ca²⁺), the formed ionically complexed calcium-carboxyl polymer in gastric acid is as stable as the other forms without complexation (e.g. sodium carboxyl polymer) as explained above. In addition, this complex possesses a higher absorbent capacity of biological fluids and is able to hydrate within the matrix maintaining the tablet integrity. This hydration within matrix with gastric acid fluid allows dissolution of active principle. The disintegrating agent contributes to the initial fast release. Simultaneously with the fast delivery of a limited amount of the active principle, the carboxyl groups involved in complexation with multivalent cations are progressively protonated, inducing thereafter the formation of a stable matrix which slows-down the release of the active principle. In intestinal environment, the matrix is gradually deprotonated (forming carboxylate sodium salts) which can interact with modulating agent (e.g. glucosamine); then the matrix will swell and/or be eroded slowly, controlling thus the release of the active principle at a slower rate.

According to one embodiment of the present invention, to modulate the duration of the initial fast release, one or more disintegrating agents are added to compositions comprising the carboxyl polymer complexed with multivalent cations of the present invention. These materials are preferably selected from insoluble substances which are pharmaceutically acceptable and compatible with a large number of active principles.

According to one embodiment of the present invention, a formulation preferably comprises a carboxyl polymer complexed with multivalent cations of the present invention and a disintegrating agent to form a matrix system that can deliver an active principle at different speeds. The characteristics of this matrix system are a fast delivery of a first effective dose of an active principle followed by a sustained release of the second effective dose over a long period of time.

According to some embodiments of the present invention, the composition may include:
Carboxyl polymer complexed with multivalent cation;
At least one disintegrating agent;
At least one modulating agent;
At least one binding agent;
At least one lubricating agent;
At least one active principle.

According to one embodiment, the matrix system of the present invention may be a monolithic tablet dosage form obtained by direct compression of the mixture of matrix and active principle powders. The tablet dosage form is able to release the active principle in two-speeds. This is a novelty for such monolithic devices that are different from those described previously for biphasic or multilayer tablets which are known in the art. Furthermore, no specific treatment of active principle, such as hot-melt extrusion of an ibuprofen/ethyl cellulose mixture (Verhoeven et al., 2006, Eur. J. Pharm. Biopharm., 63, 320-330) is necessary for preparation of monolithic tablets dosage form according to the present invention.

In embodiments, the carboxyl polymer complexed with multivalent cations of the present invention possessing carboxyl groups may be chosen from any modified polysaccharide, particularly anionic polysaccharides such as carboxymethylcellulose, carboxymethylstarch or carboxyl high amylose starch, carboxymethyl-chitosan, and the like, or natural polysaccharide such as pectin, hyaluronan (hyaluronic acid), xanthane, gellan, alginate and the like, or synthetic carboxyl polymers such as carbomer (polyacrylic acid) or cross-linked carbomer or combination thereof.

According to some embodiments, the multivalent cations that may be added to compositions comprising the carboxyl polymer complexed with multivalent cations of the present invention are preferably divalent cations such as calcium (Ca²⁺), magnesium (Mg²⁺), copper (Cu²⁺), zinc (Zn²⁺) and the like, or combination thereof.

The concentration of carboxyl polymer used in monolithic tablet form may be in the range of about 1% to about 75%, or from about 1% to about 50%, or from about 1% to about 40%, or from about 2% to about 50% or from about 2% to about 40%, or from about 5% to about 50%, or from about 5% to about 40%, and preferably about 2% to about 50% or most preferably from about 5% to about 40%.

According to embodiments of the present invention, disintegrating agents (or desintegrants) may be added to compositions comprising the carboxyl polymer complexed with multivalent cations of the present invention. Their addition in the composition serves to accelerate the tablet disintegration and dissolution, to promote release of the active principle, enhancing the bioavailability of the active principle. For this purpose, the disintegrating agents may be chosen from povidone or povidone derivatives. Preferably the povidone should be a crospovidone (or «crospolyvidone» or «cross-linked polyvidone» or «insoluble polyvidone» or «insoluble polyvinylpyrrolidone»).

Crospovidone can form chemical complexes or associate with a number of drugs and other substances possessing aromatic rings, particularly NSAIDs. Horn and Ditter (1982, J. Pharm. Sci., 71, 1021-1026) investigated aromatic compounds, in particular phenol and carboxyl groups (e.g. ibuprofen, an active ingredient possesses a benzene ring and carboxyl group) and showed that they have a strong influence on complexation. Furthermore, the degree of complexation lies within a certain range and, for most drugs, provides acceleration in dissolution rate. These interactions were also studied in hydrochloric acid and, in some cases, in simulated gastric juice according to USP (Frömming et al. 1981. J. Pharm. Sci., 70, 738-743).

The interaction of crospovidone with certain drugs can permit not only to achieve a homogenous dispersion in biological media, but also to improve drug absorption, particularly NSAIDs.

Different particle sizes of crospovidone may be used to modulate the release rate of the active principle. To obtain a moderate rate of active principle release, a relatively fine particle size of crospovidone should be used; «micronized crospovidone» is preferable. Large particles («non-micronized crospovidone») can give a more rapid release due to their greater swelling volume leading to a faster disintegration. These larges particles crospovidone are interestingly used to increase the release rate of initial dose. According to one embodiment, crospovidone as used herein may be a water uptake facilitator which may permit a first fast release (initial release) of the active principle without affecting the tablet integrity, followed by a sustained release of the active principle for a long period of time. The concentration of disintegrating agent used in monolithic tablet form may be from about 1% to about 75%, or from about 1% to about 50%, or from about 1% to about 40%, or from about 5% to about 75%, or from about 5% to about 50%, or from about 5% to about 40%, or from about 10% to about 75%, or from about 10% to about 50%, or from about 10% to about 40%, preferably about of 5% to about 50% or most preferably from about of 10% to about 40%.

According to some embodiments, modulating agents may be added to compositions comprising the carboxyl polymer complexed with multivalent cations of the present invention. The modulating agents may prolong the active principle's release. The modulating agent may be a pharmaceutically accepted compound with amino groups, such as but not limited to polyvinylpyrollidone, glucosamine, chondroitin, hyaluronate and the likes, with the role to slow-down the release of active principle. The modulating agent, with amino functional groups, may be able to interact ionically or by hydrogen binding with carboxylic groups of carboxyl polymer complexed with multivalent cations of the present invention, once the divalent cation (e.g. Ca²⁺) is released, stabilizing thus the matrix system and prolonging the active principle's release.

According to some embodiments, the modulating agent may be chosen from molecules possessing a positive charge such as glucosamine and its salts, choline, lecithin, phosphatidylcholine or amino acids such as lysine, tyrosine, glutamine, valine, phenylalanine, asparagine, arginine, leucine, isoleucine, tryptophan, histidine, methionine, threonine, serine, glycine, proline, glutamic acid, aspartic acid, cysteine, selenocysteine, and alanine, etc. or combination thereof. The modulating agent is preferably glucosamine or its salts. The modulating agent may be present in a concentration of about 1% to about 75%. or from about 1% to about 70%, or from about 1% to about 65%, or from about 1% to about 60%, or from about 1% to about 55%, or from about 1% to about 50%, or from about 1% to about 45%, or from about 1% to about 40%, or from about 1% to about 35%, or from about 1% to about 30%, or from about 1% to about 25%, or from about 1% to about 20%, or from about 1% to about 15%, or from about 1% to about 10%, or from about 1% to about 5%. The modulating agent may be present in a concentration of about 5% to about 50%, or from about 5% to about 45%, or from about 5% to about 40%, or from about 5% to about 35%, or from about 5% to about 30%, or from about 5% to about 25%, or from about 5% to about 20%, or from about 5% to about 15%, or from about 5% to about 10%. The modulating agent may be present in a concentration of about 10% to about 40%, or from about 10% to about 35%, or from about 10% to about 30%, or from about 10% to about 25%, or from about 10% to about 20%, or from about 10% to about 15%.

For different embodiments, binder agents may be added to compositions comprising the carboxyl polymer complexed with multivalent cations of the present invention. The binder agent may be used to improve the mechanical properties of tablets and favor drug release without generating a «burst effect» phenomenon during gastro-intestinal transit. It may be chosen from microcrystalline cellulose (Avicell) or cellulose derivatives such as hydroxypropyl methylcellulose (Hypromellose), Hydroxypropylcellulose (Klucel) ethylcellulose (Aqualon), amylose (Hylon), noncrosslinked polyvinylpyrrolidone (Povidone, K-value between 15-90). The use in excess of binding agent may lead to a prolonged disintegration time and decrease the rate of initial release (fast release). The prefered concentration of binding agent may be between about 0.1% to about 5%, or about 0.1% to about 10%, or about 0.1% to about 15%, or from about 0.5% to about 5%, about 0.5% to about 10%, or from about 0.1% to about 15% and preferably from about 0.5% to about 15%.

According to embodiment, a lubricating agent may be added to compositions comprising the carboxyl polymer complexed with multivalent cations of the present invention. Preferably the lubricating agent to be used may be of common mineral type like talc or silica, or fats, e.g. vegetable stearine, magnesium stearate or stearic acid or other lubricants which are commonly used in the art as lubricants in tablets. The concentration preferably used is from about 0.1% to about 0.5%, or from about 0.1% to about 1.0%, or from about 0.1% to about 1.5%, or from about 0.1% to about 2%, or from about 0.1% to about 2.5%, or from about 0.1% to about 3%, or from about 0.1% to about 3.5%.

According to embodiment, the active principle may be any suitable drug, of pharmaceutical or biological origin.

The active principle is preferably selected from drugs that provide a rapid therapeutic effect, but possess a short biological half-life, particularly NSAIDs and antihistaminic agents. Generally, NSAIDs (selective or nonselective COX-2 inhibitors) possess aromatic rings in their structure which are susceptible to complex reversibly with insoluble disintegrating agents. In this case, the release rate of active principle can indirectly be modulated via the speed of erosion afforded by the disintegrating agent.

According to some embodiment, the term “NSAID”, as used herein, represents a Non Steroidal Anti-Inflammatory Drug which can be selected from non selective or selective COX-2 inhibitors including, but not limited to:

i) Propionic acid derivatives such as Ibuprofen and/or its salts, Naproxen and/or its salts, Benoxaprofen and/or its salts, flurbiprofen and/or its salts, fenoprofen and/or its salts, fenbuprofen and/or its salts, ketoprofen and/or its salts, ioxoprofen and/or its salts, pranoprofen, carprofen, oxoprofen, microprofen, tioxaprofen, suproprofen, alminoprofen and/or fluprofen or the combination thereof.

ii) Salicylic acid derivatives such as aspirin, diflunisal, salsalate, olsalazine and sulfasalazine or the combination thereof;

iii) Acetic acid derivatives such as indomethacin, sulindac, etodolac, ketorolac, diclofenac and/or their salts or the combination thereof;

iv) Fenamic acid derivatives such as mefenamic, meclofenamic, flufenamic tolfenamic and/or their salts or the combination thereof;

iv) Selective COX-2 inhibitors such as celecoxib, valdecoxib, rofecoxib, rterocoxib, etc.

v) or combination thereof.

Several advantages of the composition are disclosed in the present invention:
Low adverse effects for patients requiring long-term NSAID therapy;
Reduced frequency of administration;
Diminished side effects related to NSAIDs especially for patients requiring long term NSAID treatments;
Lower cardiovascular risk;
Unique dose per day;

Monolithic tablet form may be easily obtained by directly compressing the mixture of active principle and matrix powders;

The present invention will be more readily understood by referring to the following examples which are given to illustrate the invention rather than to limit its scope.

The following examples further illustrate the method to produce of calcium carboxyl polymer in order to prepare the two-speed matrix system as well as the formulations of the invention. The use of particular polymers, disintegrating agents, modulating agents, binding agents or other inert components with particular amounts are not intended to limit the scope of the invention.

### EXAMPLE

### Carboxymethyl Starch Synthesis

The CarboxymethylStarch is synthesized by etherification of starch with sodium monochloroacetate under alkaline conditions. Practically, an amount of 50 g of starch, preferably high amylose starch (Hylon VII), is suspended to hydrate under stirring in 200 mL of distilled water at 60° C., and a volume of 300 mL NaOH 2 M is slowly added to the reaction medium in order to gelatinize the starch. The stirring is continued until a homogenous reaction medium is obtained. Then, a volume of 75 g of sodium monochloroacetate is rapidly dissolved in 100 mL of cold water, just prior to use, and immediately added to the reaction medium. The reaction is performed during 1 h at 60° C., always under continuous stirring. Similarly, different quantities (20-200 g) of sodium monochloroacetate are used separately in identical conditions to obtain various degrees of substitution.

At the end of the reaction, the solution is neutralized (pH 7.2) with HCl (1.0 and 0.1 M) and precipitated by adding an excess (approximately 3 L) of diluted acetone:water (60:40, v/v). The precipitated product, sodium CarboxymethylStarch (CMSNa), is collected by filtration and dehydrated 2 or 3 times with pure acetone to obtain the powder which is finally air-dried.

The carboxymethyl polymer powder can alternatively be obtained by spray-drying. This method presents several advantages such as rapidity, low cost and no need of solvents.

### EXAMPLE 2

### Analysis of CarboxymethylStarch

Fourier Transform InfraRed (FTIR) Analysis

The FTIR analysis allows to confirm that the reaction is achieved by highlighting the presence of carboxymethyl groups in the obtained powder. FTIR spectra are recorded on a Spectrum One (Perkin Elmer, Canada), instrument equipped with an UATR (Universal Attenuated Total Reflectance) device for native and CarboxymethylStarch (CMS) in powder form (20 mg), in the spectral region (4000-650 cm⁻¹) with 24 scans/min at a 4 cm⁻¹ resolution.

The results show that, after carboxymethylation of starch, new absorption bands at 1595 and 1415 cm⁻¹ appears and are assigned to carboxylate anions (asymmetric and symmetric stretching vibrations).

### EXAMPLE 3

### Determination of CarboxymethylStarch degree of substitution

The degree of substitution is determined by titrimetric method as described by Le-Tien et al. (2004. Biotechnol. Appl. Biochem., 39, 347-354) with modification as follows: the carboxymethyl groups of the CarboxymethylStarch (1.0 g) are first converted into the acidic (protonated) form by treatment of the modified polymer with a 1 M HCl solution. The protonated CarboxymethylStarch is then filtered, washed several times with distilled water in order to completely remove the acid in excess, and precipitated with pure acetone. Finally, an amount of CarboxymethylStarch is suspended in 100 mL distilled water. The carboxyl groups are titrated with a solution of NaOH 0.05 M.

Data obtained by the titration method shows that the number of carboxymethyl groups bound per glucose unit: degree of substitution, (DS) of about 0.49±0.06 (for 75 g of sodium monochloroacetate used).

At different quantities of monochloroacetate used (20-100 g), the DS are in the range 0.10.81/glucose unit.

### EXAMPLE 4

### Complexation of Carboxymethyl Starch with Calcium

An amount of 20 g of CarboxymethylStarch obtained above is dispersed in 1900 mL of distilled water under stirring at 23±1° C. until obtaining a homogenous solution. Thereafter, an excess amount of calcium chloride (about of 8 g in 100 mL of water) is added in the solution under stirring during 1.0 h. The calcium CarboxymethylStarch is obtained after precipitation with ethanol or with acetone as described for sodium CarboxymethylStarch in section 1.1. The powder is oven-dried at 40° C. during 72 h and sieved to obtain fine particles smaller than 300 μm which are used to prepare the tablets.

### EXAMPLE 5

### Structure Analysis of Calcium Carboxymethyl Starch and of Sodium CarboxymethylStarch

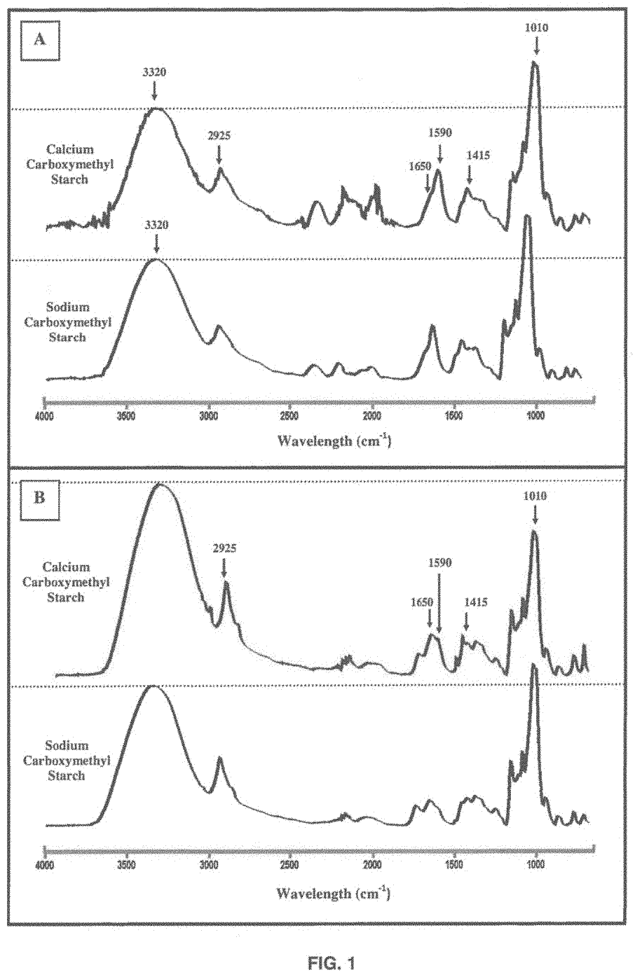

FTIR spectra analysis of sodium and calcium carboxymethyl starch, CMS(Na) and CMS(Ca) show no significant differences between the two polymers under salt forms (FIG. 1). However, when tablets are incubated 2 h in simulated gastric fluid (pH 1.5) and dried at 40° C. during 72 h, some differences are absented.

The rate of protonation of calcium CarboxymethylStarch is low and the intensities of carboxylate bands at 1590 and 1415 cm⁻¹ remain high compared to those of sodium CarboxymethylStarch. In the case of CMS(Na), the carboxylate (—COO⁻Na⁺) is protonated faster than the calcium carboxylate of CMS(Ca). Furthermore, the carboxylate of CMS(Na), when protonated, appears more stable as —COOh than the deprotonated form —COO⁻Na⁺, because the protonated form can be stabilized by polar interactions (i.e. dimerization of carboxylic acid groups) and by hydrogen associations, limiting thus the hydration.

For calcium CarboxymethylStarch, the intensity of absorption band at 3320 cm⁻¹ assigned to stretching vibrations of O—H groups is high in comparison with that of the absorption band at 1010 cm⁻¹ attributed to stretching of C—OH bonds. In contrast, this phenomenon is not observed for sodium CarboxymethylStarch (the intensity of O—H band is low compared with that of C—OH bonds). Consequently, the increase of the absorption band intensity at 3320 cm⁻¹ (O—H groups) can explain the high hydration capacity of calcium CarboxymethylStarch. The semi-maximal width, larger for CM(Na) after gastric residence, shows a higher protonation (and better hydrogen association) than in the case of CMS(Ca).

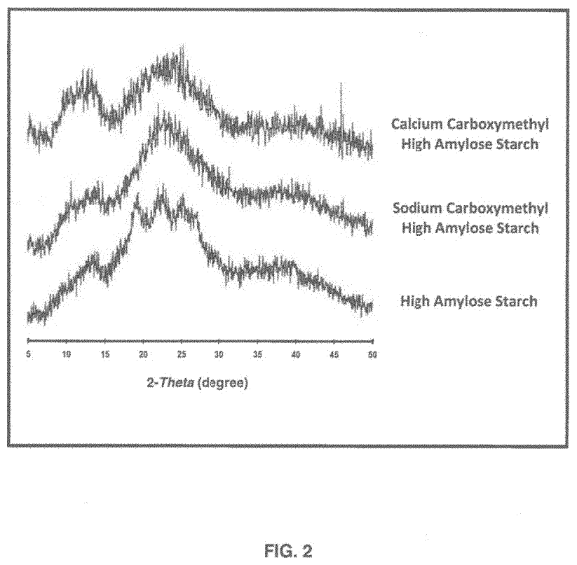

These observations fit well with the X-ray diffraction analysis. As shown in the FIG. 2, the non-modified starch (high amylose starch) possesses a structure with a higher order degree, with several crystalline domains (different helical structures). When functionalized by addition of carboxymethyl groups under sodium carboxylate form, certain domains disappear and are replaced by a single ordered band probably as V-type organization, such as reported by Assaad and Mateescu (2010, Int. J. Pharm., 394, 75-84), with similar intensity as for non-modified starch. The calcium CarboxymethylStarch shows this band even broader and with moderately low intensities, suggesting a loss of crystalline structure and its high water retention (fluid uptake) capacity. A novel band at 2θ=8° is probably related to a rearrangement due to ionic complexation with Ca²⁺.

### EXAMPLE 6

### Formulation of Monolithic Tablets Based on Calcium CarboxymethylStarch

In one embodiment, the two-speed matrix is prepared by mixing suitable quantities of calcium CarboxymethylStarch and crospovidone powders as excipients controlling the drug release according to the following recipe.

The formulation of NSAIDS monolithic tablet comprises ibuprofen, matrix (two-speed system) and lubricant agents, but could be used with other active principles:

| | | |
|---|---|---|
| Ibuprofen | 600 | mg |
| Calcium CarboxymethylStarch | 100 | mg |
| Crospovidone (micronized) | 130 | mg |
| Glucosamine | 120 | mg |
| Magnesium stearate | 20 | mg |
| Total | 970 | mg |

The formulation components are mixed in a V-blender and the resulting powders are directly compressed using conventional technologies to obtain the tablet.

### EXAMPLE 7

### Release Kinetics of Ibuprofen (600 mg) Formulated with Calcium CarboxymethylStarch

### 7.1. In Vitro Dissolution Assay

Ibuprofen in vitro release studies are conducted at 100 rpm and 37° C. using an USP paddle (apparatus II) method with a dissolution Distek 5100 (North Brunswick, N.J., USA). The ibuprofen release from tablets (Example 6, n=3) in 1 L of dissolution media is measured at 221 nm. The dissolution is realized in simulated gastric fluid (pH 1.5) for 2 h and then in enzymes-free simulated intestinal fluid (pH 6.8). At predetermined time intervals, for each sample, a volume of 2 mL is withdrawn from solution, filtered (0.20 μm) and properly diluted with simulated intestinal fluid before spectrophotometric analysis.

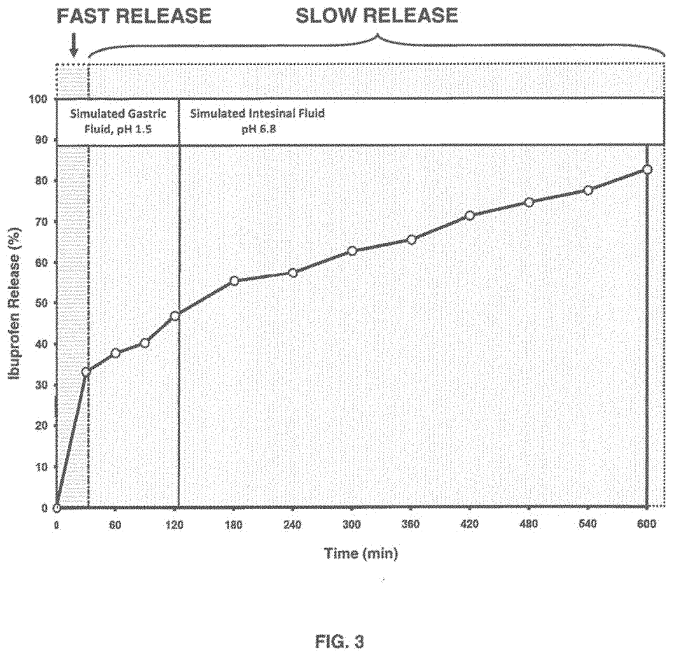

As shown in FIG. 3, the dissolution test of ibuprofen in the formulation containing calcium CarboxymethylStarch shows clearly that there are two distinct release speeds of drug release as follows:
a fast release of ibuprofen about (corresponding to the effective dose of ibuprofen that is approximately 200 mg) within 30 minutes, at 37° C. in simulated gastric fluid;
a sustained release of remaining doses for a period over 8 h.

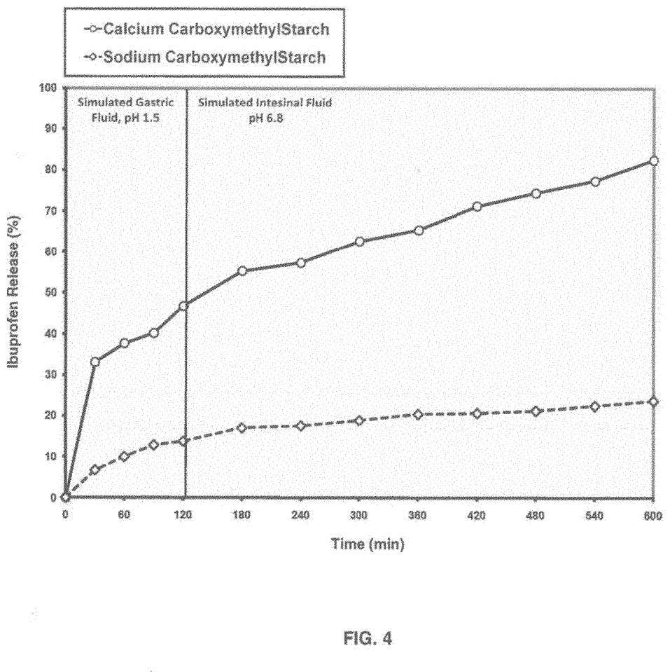

In contrast, no fast release of the effective dose of ibuprofen is observed for the formulation based-on sodium CarboxymethylStarch (FIG. 4) with a profile considered as a single sustained release rate system.

### 7.2. In vivo Study on Dogs (Canis familiaris)

The main objective is to compare the pharmacokinetic parameters of the calcium CarboxymethylStarch formulation (as described in Example 6) with a conventional form of ibuprofen (Motrin®) after oral administration of tablet samples.

### 7.2.1. Preparation of Tablets

Preparation of Ibuprofen matrix free

| | |
|---|---|
| Ibuprofen USP | 200 mg |

directly compressed using conventional technologies to obtain the tablet.

Preparation of ibuprofen controlled release monolithic tablets:

| | | |
|---|---|---|
| Ibuprofen USP | 400 | mg |
| Calcium carboxymethylstarch | 80 | mg |
| Crospovidone (micronized) | 93 | mg |
| Glucosamine•HCl | 80 | mg |
| Magnesium stearate | 13 | mg |
| Total | 666 | mg |

Motrin®:

Motrin® tablets (200 mg Ibuprofen) are obtained commercially and used as received.

### 7.2.2. Subjects and Study Design

This in vivo study is carried out on the male dogs (Canis familiaris, body weight approximately 10.7 kg) and the experimental protocol is conducted according to the Animal Care Committee (INRS-Institut Armand-Frappier, Centre de Biologie Expérimentale, Laval, Québec, Canada) and is approved before the experiment.

After reviewed the medical records provided by the supplier animal facility (Marshall Bioresources, North Rose, N.Y.), a re-evaluation of the dog health condition by the veterinarian is done. To conform to the Animal Care Committee recommendations, the dogs are observed during one week for the acclimatation period before experiment.

Three groups (n=4 dogs/groups) are subjected to treatments as follows:

Group-1 (n=4 dogs): monolithic tablets containing 200 mg of ibuprofen (matrix-free) administered every 4 h (1 tablets/dog);

Group-2 (n=4 dogs): monolithic tablets containing 400 mg of Ibuprofen and the calcium CarboxymethylStarch formulation administered 1 time (1 tablet/dog);

Group-3 (n=4 dogs): commercial Motrin® immediate release containing 200 mg of Ibuprofen administered every 4 h (totally, 3 tablets/dog).

After receiving treatments by oral administration, blood samples are collected in heparinized tubes without anesthesia. In fact, an amount of 1.6 mL of blood samples are collected prior to treatment (pre-dose t=0) and at the following times post-dose: 0.5, 1.0, 1.5, 2.0, 2.5, 3.0, 3.5, 4.0, 6.0, 8.0, 10.0, 12.0 and 24 h. The Ibuprofen concentrations are determined using a liquid chromatography with tandem mass spectrometry (LC-MS-MS) method (a validated assay method by Eliapharma Services Inc., Laval, Quebec, H7V 4A9, Canada).

After blood sampling and centrifugation, the obtained plasma is subjected to an extraction and quantification procedure as follows:

### 7.2.3. Extraction of Ibuprofen

in an Eppendorf® tube, it is vortexed 50 μL of plasma with 20 μL of 100 μg/mL Ibuprofen-d3 (Internal Standard) and 280 μL phosphoric acid 4% (v/v);

spun in centrifuge (Eppendorf® 5414 Bench-Top Centrifuge) for 2000 rpm during 1 minute;

washed once time in 1 mL of Methanol 5% and another time with 0.5 mL in the same solution methanol for 1 minute;

transfered the eluted solution into 96-well plate for further analysis.

### 7.2.4. Quantification of Ibuprofen

The determination of Ibuprofen concentration in plasma extract is carried out by the LC-MS-MS analytical method with a CBM-20A controller, DGU-14A and 20A online degassers, LC-10A ©VP and LC-20AD pumps (Shimadzu, Tokyo, Japan) with a pre-column Zorbax™ Eclipse XDB-C8 (2.1×12.5 mm, 5 μm) and analytical columns Zorbax™ SB-C18 (2.1×50 mm, 3.5 μm) and Zorbax™ XDB-C18 (3.0×150 mm, 5 μm; Agilent Technologies®, CA, USA). The Ibuprofen standard curve is prepared just prior to each analysis using sodium Ibuprofen-d3 (C/D/N Isotopes Inc., Qc, CA) as internal standard.

The chromatographic separation is achieved at ambient temperature using the mobile phase consisting in acetonitrile : water (6:4) with pH adjusted to 2.6 with phosphoric acid at a flow rate of 0.4 mL/min. The injection volume is 20 μL and the total cycle run time including equilibrium time is 5.5 minutes (4.5 minutes run time+1 minute for injection). All solvents used are HPLC grade from Fisher Scientific®.

For Mass Spectrometry, the model is API-4000 from Applied Biosystem® (CA, USA) operated in selected reaction monitoring (SRM) mode with negative electrospray ionization. The ibuprofen and ibuprofen-D3 SRM transitions with mass to charge (m/z) ratios are m/z 205.4→161.2 and m/z 208.2→164.2, respectively.

The pharmacokinetic parameters are calculated by using Thermo Kinetica™ software version 5.0. Ibuprofen plasma concentration/times are analyzed using no compartmental pharmacokinetics. This approach is highly dependent on the estimation of total drug exposure. The parameters calculated are extrapolated plasma concentrations:

peak plasma concentration (Cmax);

time to reach the peak plasma concentration (Tmax);

area under the concentration-time curve from time zero to last quantifiable concentration (AUC₀₋ₜ);

area under the concentration-time curve from time zero to infinity (AUC_{0-∞});

elimination half-life (T1/2);

Ke=terminal elimination rate per hour;

mean residence time (MRT).

Twelve dogs are randomly divided in three groups (n=4 dogs per group), corresponding to the formulations previously described in the section 7.2.2. Subjects and Study Design.

During the time-point sampling, each dog is observed for any signs of distress or excessive stress. Following these minor manipulations, all of the dogs are physically and clinically healthy after experiments.

### 7.2.5. Hematology and Urine Analysis

Blood sampling for hematology is taken at time 0 h predose and at 24.0 h postdose and a hematology test including cells counts (i.e., WBC, RBC, hemoglobin, hematocrit, MCV, MCH, MCHC, reticulocytes, and platelets), a differential count (Le., bands, neutrophils, lymphocytes, monocytes, eosinophils, and basophils), and cells morphology (i.e., WBC, RBC, and platelets) is carried out for each sample. No abnormal signs are observed for each dog (before and after experience).

Urine samples are also collected during the experiment and analyzed with a Multistix® 10SG. The objective is to check eventual toxic signs after the experiment. The urinary samples are taken before the exposure and compared to that at 24.0 hours post-exposure. No difference is observed for these analyses and the results suggest that no Ibuprofen formulations in the experience can cause a toxicity.

### 7.2.6. In Vivo Results

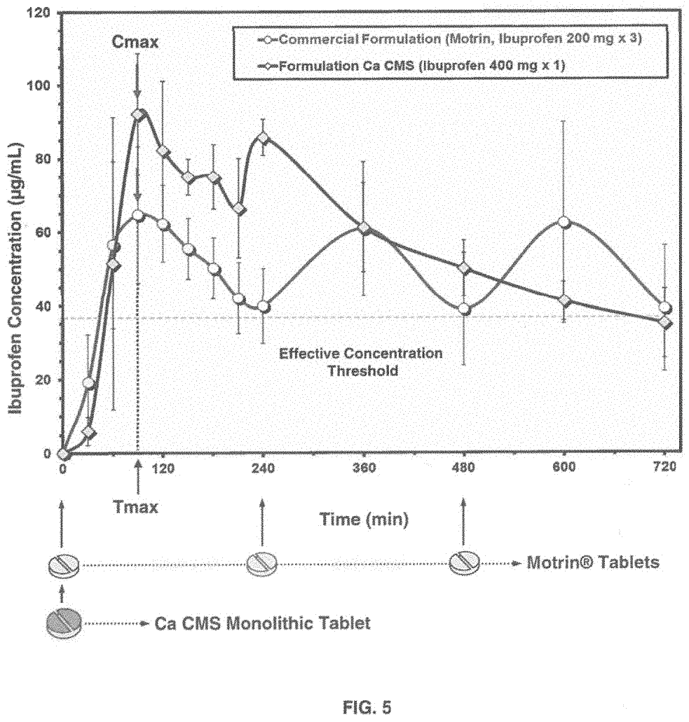

The main objective of the in vivo study consists in evaluating the first immediate release followed by the extended release of Ibuprofen 400 mg single dose formulated with complex Ca CMS compared with Motrin® 200 mg×3 tablets immediate-release tablets (an over-the-counter reference). The quantification of plasma Ibuprofen shows that the Cmax (92 μg/mL) from the new controlled-release formulation possesses a value superior to Cmax (65 μg/mL) of Motrin immediate release (FIG. 5). Crospovidone can be used not only as a disintegrating agent, but also to improve the bioavailability of Ibuprofen. This explains why Cmax obtained from the new controlled-release formulation is higher than that from Motrin®. However, the statistical analysis of Cmax in this exploratory study showed no significant difference.

In view of Tmax (1.30 h) values, there is no significant difference between the new controlled-release formulation and Motrin® immediate release. The Tmax value is of about 1.5 h (FIG. 5).

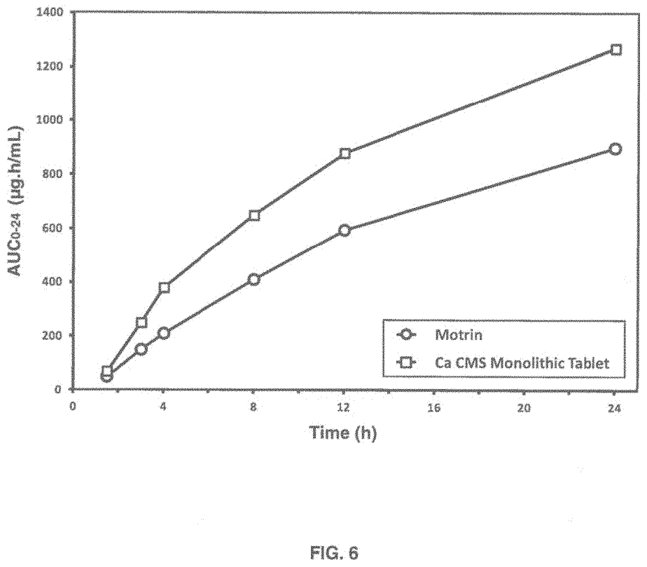

More interestingly, the AUC₀₋₂₄ₕ value (981 μg.h/mL) of the new controlled-release formulation containing 400 mg Ibuprofen×1 tablet closely matched the AUC₀₋₂₄ₕ value (899 μg.h/mL) obtained for the Motrin® 200 mg×3 tablets. Other detailed parameters are presented in the Table 1 and FIG. 6.

TABLE 1
Pharmacokinetic parameters in Beagle dog of Ibuprofen formulated
with complex Ca CMS and commercial Motrin ®

| | | Groups | | |
|---|---|---|---|---|
| Parameter | Unit | 1 | 2 | 3 |
| Test or control | | Ibuprofen | Ibuprofen | Motrin ® |
| articles | | Matrix-Free | Monilithic | |
| | | | Tablet | |
| | | | formulated | |
| | | | with Ca CMS | |
| Dose | mg | 200 | 400 | 200 |
| Number of dose | | 1 | 1 | 3 (every |
| | | | | 4 h, at t0, |
| | | | | t4 and t8) |
| Route of | | Oral | Oral | Oral |
| administration | | | | |
| Cₘₐₓ | μg/mL | 29 | 92 | 65 |
| Tₘₐₓ | h | N/A | 1.5 | 1.5 |
| AUC_{0-24 h} | μg · h/mL | 399 | 981 | 899 |
| AUC_{0-∞} | μg · h/mL | N/A | 1352 | 1022 |
| T_{1/2} | h | N/A | 9.9 | 7.1 |
| Ke | 1/h | 0.272 | 0.070 | 0.098 |
| MRT | h | 3.7 | 15.1 | 12.2 |

Legend:
Cmax = maximal concentration;
Tmax = time at maximal concentration;
AUC_{0-∞} = area under the concentration-time curve from time zero to infinity;
AUC_{0-24 h} = area under the concentration-time curve from time zero to 24 hours;
T_{1/2} = elimination half-life;
Ke = terminal elimination rate per hour;
MRT = mean residence time.

Generally, the in vivo study on beagle dogs shows that pharmacokinetic parameters of the new controlled release formulation for single dose ibuprofen (400 mg) are near equivalence with multiple doses (3 tablets of 200 mg Ibuprofen) of conventional formulation Motrin®. In this case, Ibuprofen formulated with new controlled released formulation allows to:
Provide effective concentrations similar to that obtained with the conventional forms (Motrin®) required for rapid pain relief;
Deliver the drug initially in the stomach at a rate similar to that obtained with the conventional forms, and to maintain effective drug concentrations for a longer period of time after a single dose required for sustained chronic pain relief and other anti-inflammatory effects.
Maintain a serum concentration after a single dose similar to those achieved after repeated dosing with the conventional form.

Furthermore, the new controlled release formulation allows to reduce the absorption of an amount of Ibuprofen while maintaining an effective concentration in the blood, similar to that of the multiple doses of conventional form. This reduction of amount is important since it eliminates or diminishes side-effects associated with NSAIDs, particularly in decreasing the risk of cardiovascular diseases.

### EXAMPLE 8

### Release Kinetics of Ibuprofen from Calcium CarboxymethylStarch with Various Degree of Substitution

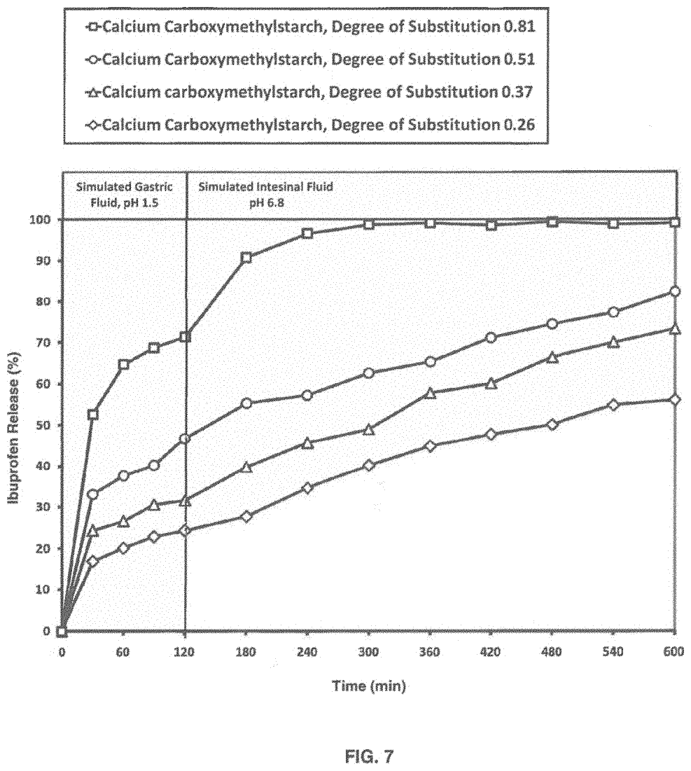

Monolithic tablets based on calcium CarboxymethylStarch with different degrees of substitution (DS 0.19-0.81) are prepared as described in example 6. The results of dissolution tests (FIG. 7) show that the kinetic profiles are different for carboxylated polymers possessing different DS. The rate and the amount of initial drug release are different at various DS of CarboxymethylStarch.

The drug release kinetics from calcium CarboxymethylStarch with DS 0.81 show a faster release: about 50% with a shorter time of sustained release. At this high degree of substitution, the CMS becomes insoluble after complexation with calcium ions (ionotropic gelation). In the preferred embodiment, a partial complexation of carboxymethyl polymers possessing a high carboxylation degree (DS) is chosen, using suitable concentrations of multivalent cations (e.g. calcium ion). In this case, the CarboxymethylStarch (high DS) partially complexed with calcium remains soluble and gives the same release kinetics comparable with those at low degree of substitution.

For this purpose, an amount of 20 g of sodium CarboxymethylStarch with a high degree of substitution (DS higher than 0.8) are dispersed under stirring in 1900 mL of distilled water until a homogenous solution is obtained. Then, a quantity of calcium chloride (about 4.2 g in 100 mL of deionized water) is added to the solution under stirring during 1.0 h. The powder of CarboxymethylStarch partially complexed with calcium is obtained after precipitation in ethanol or in acetone. The drying is realized as described for sodium CarboxymethylStarch in the example 1.

### EXAMPLE 9

### Formulations Based on Calcium CarboxymethylStarch Composed with Different NSAIDs

The following examples of formulation are non-limiting examples of preferred formulations:

1. Formulations composed with Acetylsalicylic acid

| | | |
|---|---|---|
| Acetylsalicylic acid | 500 | mg |
| Calcium carboxymethylstarch | 120 | mg |
| Crospovidone (micronized) | 75 | mg |
| Kollidon | 120 | mg |
| Microcrystalline cellulose | 50 | mg |
| Magnesium stearate | 25 | mg |
| Total | 890 | mg |

2. Formulations composed with Diflunisal

| | | |
|---|---|---|
| Diflunisal | 500 | mg |
| Calcium carboxymethylstarch | 170 | mg |
| Crospovidone (micronized) | 70 | mg |
| Kollidon | 70 | mg |
| Magnesium stearate | 10 | mg |
| Total | 820 | mg |

3. Formulations composed with Indomethacine

| | | |
|---|---|---|
| Indomethacin | 150 | mg |
| Calcium carboxymethylstarch | 85 | mg |
| Crospovidone (micronized) | 10 | mg |
| Glucosamine | 20 | mg |
| Microcrystalline cellulose | 3 | mg |
| Magnesium stearate | 2 | mg |
| Total | 270 | mg |

Production of Calcium Carboxymethylcellulose and Composition of Two Speed Matrices for Controlled Release of NSAIDs (Ibuprofen, Acetyl Salicylic Acid, Diflunisal, Indomethacin)

In a preferred embodiment, calcium carboxymethylcellulose can be produced from sodium carboxymethylcellulose.

### EXAMPLE 10

### Complexation of Carboxymethylcellulose with Calcium

The ionic complexation process is similar to that described in Example 3. An amount of 20 g of carboxymethylcellulose is dispersed in 1900 mL of distilled water under stirring at 23±1° C. until obtaining a homogenous solution. Then, an excess quantity of calcium chloride (about of 8 g in 100 mL of water) is added to the solution under stirring during 1.0 h, for complexation. The powder of calcium carboxymethylcellulose is obtained after precipitation in ethanol or in acetone and is oven-dried at 40° C. during 72 h.

### EXAMPLE 11

### Structure Analysis of Calcium and Sodium Carboxymethylcellulose

Similarly as for CarboxymethylStarch, no evident differences of FTIR spectra are noticed for the both complexed polymers, CMC(Na) and CMC(Ca), under salt or protonated forms. However, after incubation for 2 h in simulated gastric fluid (pH 1.5) and after the drying procedure, the intensity of absorption band of O—H groups (3365 cm⁻¹) is higher for calcium carboxymethylcellulose compared to that of sodium carboxymethylcellulose, suggesting a higher fluid retention of the polymer calcium salt (a stronger hydrogen association of hydroxylic groups for sodium salt) (FIG. 6). Furthermore, X-ray diffraction analysis (FIG. 7) shows a higher order degree of sodium carboxymethylcellulose (structure more crystalline and more organized) than that of calcium carboxymethylcellulose. These X-ray data fit well with the FTIR observations.

### EXAMPLE 12

### Release Kinetic Profiles of Ibuprofen (600 mg) Formulated with Calcium Carboxymethylcellulose

Ibuprofen in vitro release studies were conducted as described above for calcium CarboxymethylStarch in the examples 6 and 7. A similar profile is observed as that with calcium CarboxymethylStarch: the dissolution test of ibuprofen formulated with calcium carboxymethylcellulose shows clearly two distinct speeds of drug release:
a fast release of about 38.5% ibuprofen over the first 60 min, at 37° C. in simulated gastric fluid;
a sustained release of remaining doses during the subsequent 6 h.

In contrast, no fast release of the effective dose of ibuprofen is observed for the formulation based-on sodium carboxymethylcellulose which is considered as a single sustained release rate.

### EXAMPLE 13

### Production of Calcium Pectin as Excipient and Composition of a Two Speed Matrix for Controlled Release of Ibuprofen

Pectin represents a type of natural polysaccharide that contains galacturonic acid residues which can be in esterified or non-esterified forms. In the preferred embodiment, calcium pectate or calcium pectinate or calcium pectin is preferably used.

The main advantage of the use of natural carboxyl polymers is the already constant number of carboxyl groups, instead of synthesizing or chemically functionalizing polymers by carboxymethylation.

In this example, the available carboxyl groups are those remained under non-esterified forms. To increase the number of carboxyl groups, a simple treatment of the pectin for about 1-3 days under alkaline conditions or with specific enzymes (pectinases) is necessary.

### EXAMPLE 14

### Complexation of Pectin with Calcium

The ionic complexation process is similar to that described in example 4. An amount of 20 g of pectin is dispersed in 1900 mL of distilled water and the pH of solution is adjusted at 7.2. Thereafter, a quantity of calcium chloride (about 10 g in 100 mL of water) is added under stirring to the solution during 1.0 h. The powder of calcium pectate is obtained by precipitation in ethanol or acetone and oven-dried at 40° C. during 72 h.

### EXAMPLE 15

### Release Kinetics of Ibuprofen Formulated with Calcium Pectate

Formulations of Calcium Pectate Composed with Ibuprofen

| | | |
|---|---|---|
| Ibuprofen | 600 | mg |
| Calcium pectate | 140 | mg |
| Crospovidone (micronized) | 90 | mg |
| Glucosamine | 100 | mg |
| Hydoxypropyl methylcellulose | 15 | mg |
| Magnesium stearate | 5 | mg |
| Total | 950 | mg |

Release Kinetics of Ibuprofen from Calcium Pectate

The monolithic tablets of Ibuprofen formulated with calcium pectate show an initial fast release of about 25% followed by a sustained release over the next 7 h.

Production of Calcium Hyaluronate and Composition

Hyaluronate (also called hyaluronic acid or hyaluronan) is a natural anionic polysaccharide (non-sulfated): a polyglycoaminoglycan containing carboxyl groups. In a preferred embodiment, calcium hyaluronate is preferably used.

### EXAMPLE 16

### Complexation of Hyaluronate with Calcium

The ionic complexation process is similar to that described in example 3. An amount of 20 g of sodium or potassium hyaluronate is dispersed in 900 mL of distilled water. Then, an amount of calcium chloride (about 10 g in 100 mL of water) is added to the solution, under stirring, for 1.0 h. The powder of calcium hyaluronate is obtained by precipitation in ethanol or in acetone and the residue obtained on the filter is oven-dried at 40° C. for 48-72 h.

### EXAMPLE 17

### Release Kinetic of Ibuprofen Formulated with Calcium Hyaluronate

Formulations of Calcium Hyaluronate with Ibuprofen for Monolithic Tablets

| | | |
|---|---|---|
| Ibuprofen | 600 | mg |
| Calcium hyaluronate | 90 | mg |
| Crospovidone (micronized) | 140 | mg |
| Kollidon | 30 | mg |
| Hydoxypropyl methylcellulose | 30 | mg |
| Magnesium stearate | 10 | mg |
| Total | 870 | mg |

Release Kinetics of Ibuprofen from Calcium Hyaluronate

Monolithic tablets of Ibuprofen formulated with calcium hyaluronate presented an initial fast release of about 35% of the drug and then a sustained release over 7 h. It is worth to mention that the combination of drug (e.g. ibuprofen) and its salt (e.g. sodium ibuprofenate) could increase the initial release rate.

While preferred embodiments have been described above and illustrated in the accompanying drawings, it will be evident to those skilled in the art that modifications may be made without departing from this disclosure. Such modifications are considered as possible variants comprised in the scope of the disclosure.

1. A dosage form having a monolithic matrix for delivery of an active ingredient at two different release rates comprising:
a complex of non-modified carboxyl high amylose-starch with calcium;
a disintegrating agent selected from the group consisting of a crosslinked povidone derivative, a crospovidone, a crosslinked sodium carboxymethyl cellulose, a crosslinked starch, a cross-linked starch glycolate, a crosslinked cellulose, a crosslinked cellulose derivative, a crosslinked alginate, a crosslinked soy polysaccharide, and combinations thereof, for a first initial fast release of said active ingredient; and
a modulating agent, for a second sustained release of said active ingredient.

2. The dosage form according to claim 1, wherein the non-modified carboxyl high amylose-starch complexed with calcium is present in a concentration of about 1% to about 75% w/w.

3. The dosage form according to claim 1, wherein the disintegrating agent is present in a concentration of about 1% to about 75% w/w.

4. The dosage form according to claim 1, wherein said modulating agent is selected from the group consisting of a polyvinylpyrollidone, a chondroitin, a hyaluronate, a molecule containing an amino group, and combinations thereof.

5. The dosage form according to claim 4, wherein said molecule containing an amino groups is selected from the group consisting of a glucosamine, an oligochitosane, a lecithin, a choline, an amino acid and combinations thereof.

6. The dosage form according to claim 4, wherein said modulating agent is present in a concentration from about 1% to about 75% w/w.

7. The dosage form according to claim 1, further comprising a binder agent.

8. The dosage form according to claim 7, wherein said binder agent is selected from the group consisting of a microcrystalline cellulose, hydroxypropyl methylcellulose, hydroxypropylcellulose, ethylcellulose, methyl cellulose, amylose, and combinations thereof.

9. The dosage form according to claim 7, wherein the binder agent is present in a concentration of about 0.1% to about 15% w/w.

10. The dosage form according to claim , further comprising a lubricating agent.

11. The dosage form according to claim 10, wherein said lubricating agent is selected from the group consisting of talc, silica, a fat, sorbitol, a polyethylene glycol (PEG), and combinations thereof.

12. The dosage form according to claim 11, wherein the lubricating agent is present in a concentration of about 0.1% to about 3.5%.

13. The dosage form according to claim 1, further comprising an active ingredient.

14. The dosage form according to claim 13, wherein said active ingredient is selected from the group consisting of a non-steroidal anti-inflammatory drug (NSAID) and an antihistaminic agent.

15. The dosage form according to claim 2, wherein the carboxyl polymer complexed with a multivalent cation is present in a concentration of about 2% to about 50% w/w.

16. The dosage form according to claim 1, wherein the disintegrating agent is present in a concentration about 5% to about 50% w/w.

17. The dosage form according to claim 4 wherein said modulating agent is present in a concentration from about 5% to about 50% w/w.
